(19) United States
(12) Patent Application Publication
(10) Pub. No.: US 2019/0239724 A1
(43) Pub. Date: Aug. 8, 2019

(54) DEVICES FOR USE IN INTERVENTIONAL AND SURGICAL PROCEDURES AND METHODS OF USE THEREOF

(71) Applicant: Body Vision Medical Ltd., Ramat Hasharon (IL)

(72) Inventors: Dorian Averbuch, Ramat Hasharon (IL); Eliron Amir, Ramat Hasharon (IL); Boaz Harari, Ramat Hasharon (IL)

(21) Appl. No.: 16/339,209

(22) PCT Filed: Oct. 5, 2017
(86) PCT No.: PCT/IB2017/001376

§ 371 Date: Apr. 3, 2019

Related U.S. Application Data
(60) Provisional application No. 62/405,673, filed on Oct. 7, 2016.

(51) Int. Cl. A61B 1/00; A61B 1/267; A61B 5/06; A61M 25/09; A61B 34/20

(52) U.S. Cl. CPC A61B 1/00087; A61B 1/00101; A61B 1/00131; A61B 2576/02; A61B 5/065; A61M 25/09; A61B 34/20; A61B 1/2676

(57) ABSTRACT

A device including an applicator having a proximal end, a distal end, an internal channel extending therebetween; a shaft having a proximal end, a distal end, and an internal channel extending therebetween, the shaft being configured to be slidably received within the internal channel of the applicator; a catheter configured to be positioned within the internal channel of the shaft; a guide wire positioned within the catheter; a connector configured to be attached to the distal end of the applicator, to engage a bronchoscope, and so as to be rotatable with respect to the shaft; a handle attached to the proximal end of the applicator, the handle including a trigger operable to selectively lock or unlock sliding motion of the shaft with respect to the applicator; and a radio opaque material attached to an outer portion of the device and positioned in a predetermined pattern.

### CROSS-REFERENCE TO RELATED APPLICATIONS

This application is an international (PCT) application relating to and claiming the benefit of commonly-owned, copending U.S. Provisional Patent Application No. 62/405,673, entitled “DEVICE FOR USE IN SURGICAL PROCEDURES AND METHODS OF USE THEREOF,” filed Oct. 7, 2016, the contents of which are incorporated by reference herein in their entirety.

### FIELD

The present invention relates to medical imaging. More particularly, the present invention relates to a device that is configured to attach to a distal end of a bronchoscope, to enable navigation of the device when the device is positioned within a patient's body, and to enable determination of the depth of the device based on a two-dimensional medical image showing the device positioned within the patient's body. The present invention also relates to a method for using such a device.

### BACKGROUND

Bronchoscopes are medical devices that are used to obtain images of body cavities within the body of a patient (e.g., within a patient's lung). To properly evaluate the images obtained using a bronchoscope, the position of the bronchoscope in three dimensions (i.e., including the depth of the bronchoscope within the body) must be known.

### SUMMARY

In an embodiment, a device configured to be attached to a bronchoscope includes an applicator, a shaft, a catheter, a guide wire, a connector, a handle, and a radio opaque material, the applicator having a proximal end, a distal end, and an internal channel extending from the proximal end to the distal end, the shaft having a proximal end, a distal end, and an internal channel extending from the proximal end to the distal end, the shaft being configured to be slidably received within the internal channel of the applicator, the catheter configured to be positioned within the internal channel of the shaft, the guide wire positioned within the catheter, the connector configured to be attached to the distal end of the applicator, configured to engage a bronchoscope, and configured so as to be rotatable with respect to the shaft, the handle attached to the proximal end of the applicator, the handle comprising a trigger operable to selectively lock or unlock sliding motion of the shaft with respect to the applicator, the radio opaque material attached to an outer portion of the device, the radio opaque material being positioned in a predetermined pattern.

In an embodiment, the pattern is non-uniform. In an embodiment, the pattern includes the radio opaque material having a first density at a first location and a second density at a second location, the first and second densities being different from one another. In an embodiment, the radio opaque material is positioned (a) on the catheter, (b) on the guide wire, or (c) on both the catheter and the guide wire.

In an embodiment, the proximal end of the applicator includes a luer lock entrance. In an embodiment, the connector includes a luer lock plug that is connected to the luer lock entrance of the proximal end of the applicator.

In an embodiment, the guide wire is either flexible, rigid, pre-curved, and or configured to be curved. In an embodiment, the catheter includes a pull wire that is configured to control a curvature of the guide wire. In an embodiment, the grip handle is configured to rotate with respect to the shaft. In an embodiment, the device also includes a polytetrafluoroethylene tube positioned within the shaft and configured to guide movement of the catheter.

In an embodiment, a method for medical imaging includes providing a bronchoscope; the method also including providing a device configured to be attached to the bronchoscope, the device including an applicator, a shaft, a catheter, a guide wire, a connector, a handle, and a radio opaque material, the applicator having a proximal end, a distal end, and an internal channel extending from the proximal end to the distal end, the shaft having a proximal end, a distal end, and an internal channel extending from the proximal end to the distal end, the shaft being configured to be slidably received within the internal channel of the applicator, the catheter configured to be positioned within the internal channel of the shaft, the guide wire positioned within the catheter, the connector configured to be attached to the distal end of the applicator, configured to engage a bronchoscope, and configured so as to be rotatable with respect to the shaft, the handle attached to the proximal end of the applicator, the handle comprising a trigger operable to selectively lock or unlock sliding motion of the shaft with respect to the applicator, the radio opaque material attached to an outer portion of the device, the radio opaque material being positioned in a predetermined pattern; the method also including attaching the device to the bronchoscope; the method also including placing the bronchoscope within a body cavity of a body of a patient; the method also including obtaining at least one medical image of at least a portion of the body of the patient, the at least a portion including the body cavity; and the method also including determining a depth of the device within the body based on at least the predetermined pattern and the at least one medical image.

In an embodiment, the medical image is an X-ray.

### BRIEF DESCRIPTION OF THE DRAWINGS

FIG. 1 shows a flowchart of an exemplary method.

FIG. 2A shows a plot of density of radio opaque material along the length of an exemplary device.

FIG. 2B shows a plot of grayscale intensity in a fluoroscopic image of the device of FIG. 2A.

FIG. 2C shows a plot of grayscale intensity in a fluoroscopic image of the device of FIG. 2A with the device partially occluded.

FIG. 2D shows the correlation between the grayscale intensity of the imaged device and the density of radio opaque material.

FIG. 2E shows a rendering of an exemplary device including a pattern of radio opaque material as positioned in a patient's lung and partially occluded.

FIG. 2F shows a chart of a first exemplary pattern of radio opaque material on an exemplary device.

FIG. 2G shows a rendering of an exemplary device including a pattern of radio opaque material as positioned in a patient's lung and partially occluded, the device having radio opaque material of a density as shown in FIG. 2A.

FIG. 2H shows exemplary rings of radio opaque material of varying size and varying spacing along the length of an exemplary device.

FIG. 2I shows a chart of a second exemplary pattern of radio opaque material on an exemplary device.

FIG. 3A shows an exemplary device including an applicator, a catheter, and a guide wire, the device being shown disassembled.

FIG. 3B shows the applicator of FIG. 3A in an extended position.

FIG. 3C shows the applicator of FIG. 3A in a retracted position.

FIG. 4A shows the device of FIG. 3A, the device being shown assembled.

FIG. 4B shows the device of FIG. 4A, the device being shown with a guide wire extended.

FIG. 5 shows an exploded view of the applicator shown in FIG. 3A.

FIG. 6A shows the applicator of FIG. 3A, a trigger of the applicator being shown in an unlocked position.

FIG. 6B shows the applicator of FIG. 3A, a trigger of the applicator being shown in a locked position.

FIG. 7A shows a partial sectional view of the applicator shown in FIG. 6A.

FIG. 7B shows a partial sectional view of the applicator shown in FIG. 6B.

FIG. 8A shows a portion of the applicator of FIG. 3A, the applicator being viewed from the opposite direction from that shown in FIG. 3A.

FIG. 8B shows a partial sectional view of the applicator of FIG. 3A.

FIG. 9A shows the exemplary assembled device of FIG. 4A, the applicator of the device being shown in an extended position and in proximity to disengaged connector portions.

FIG. 9B shows the exemplary assembled device of FIG. 4A, the distal portion of the shaft being shown in proximity to a removable connector portion.

FIG. 10 shows an exploded view of an exemplary shaft of the exemplary applicator of FIG. 3A.

FIG. 11A shows a sectional view of an exemplary wire extraction button of the exemplary applicator of FIG. 3A.

FIG. 11B shows an exploded view of the exemplary wire extraction button of FIG. 11A.

FIG. 12 shows a sheath luer lock entrance of the exemplary applicator of FIG. 3A.

FIG. 13A shows an exemplary luer lock plug that is configured to engage an exemplary connector of the applicator of FIG. 3A.

FIG. 13B shows the exemplary luer lock plug of FIG. 13A engaging the exemplary connector of the applicator of FIG. 3A.

### DETAILED DESCRIPTION

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the term “radio opaque” refers to a material that is characterized in that electromagnetic radiation (including, but not limited, to X-rays) is unable to pass through such a material.

In some embodiments, the present invention is a device, comprising:

an applicator;
a shaft;
a catheter;
a guide wire;
a connector;
a handle;
a trigger;
a luer lock plug; and
a radio opaque material;

wherein the applicator has an inner open channel from a proximal end to a distal end of the applicator,
wherein the inner open channel of the applicator is of a sufficient size to house the shaft;
wherein the shaft is of a sufficient size to house the catheter and the guide wire,
wherein the catheter and the guide wire are configured to have an extraction button which allow the guide wire to protrude out the catheter,
wherein the catheter and the guide wire are configured to have pre-curved distal tip
wherein the catheter proximal end is configured to have a luer lock entrance
wherein the guide wire is configured to be connected or detached from the catheter,

wherein the shaft is configured allow displacement inside and outside the applicator,

wherein the shaft distal end is configured to allow the shaft to rotate,

wherein the shaft distal end is configured to be connected or detached from the connector,

wherein the distal end of the applicator is attached to the connector,
wherein the connector is configured to attach to a bronchoscope,
wherein the connector is configured to be connected or detached from the bronchoscope,
wherein the connector is configured to include a luer lock plug,

wherein the proximal end of the applicator is attached to the handle,
wherein the handle comprises a switch configured to lock and unlock the handle,
wherein the handle is configured to rotate from an open position to a closed position,
wherein the shaft is configured to rotate with the handle, and
   wherein the radio opaque material is attached to an outer portion of the device.

In some embodiments, the radio opaque material is dispersed in a pattern.

In some embodiments, the pattern is not uniform.

In some embodiments, the dispersed pattern comprises a plurality of deposited densities of the radio opaque material on the outer portion of the device.

In some embodiments, a first deposited density of the deposited densities is not identical to a second deposited density of the deposited densities.

In some embodiments, the pattern comprises at least one shape.

In some embodiments, the at least one shape can be a ring.

In some embodiments, the ring can be an unbroken ring.

In some embodiments, the ring can be a broken ring.

In some embodiments, the pattern is in a longitudinal conformation in reference to the applicator.

In some embodiments, the grip handle is free to rotate with respect to the shaft. In some embodiments, the grip handle is constrained from rotation with respect to the shaft. In some embodiments, the grip handle is selectively either free to rotate with respect to the shaft or constrained from rotation with respect to the shaft. In some embodiments, the selective freedom or restriction of rotation of the grip handle with respect to the shaft is independent from restriction of longitudinal motion of the shaft.

In some embodiments, the guide wire is curved.

In some embodiments, the catheter is curved.

In some embodiments, the catheter has a pull wire allowing the curvature of the distal end of the catheter to be manipulated.

In some embodiments, the shaft includes a mechanism allowing rotation of the handle to be controlled.

In some embodiments, the device includes a locking mechanism configured to selectively lock or unlock movement of the catheter along a longitudinal axis of the device, while allowing the catheter to rotate about the longitudinal axis.

In some embodiments, the shaft includes a groove that allows the catheter to be inserted along the side of the shaft.

In some embodiments, the device includes a polytetrafluoroethylene tube located inside the shaft so as to hold the catheter and guide the catheter outside the shaft.

In some embodiments, the guide wire can be extracted from the catheter by demand in order to control the effective curvature of the distal tip of the device. In some embodiments, the device includes a manipulator that is configured to control the motion of the guide wire.

In some embodiments, the guide wire can be detached from the catheter.

In some embodiments, the catheter can be detached from the handle.

In some embodiments, the handle is configured to be detached from the connector without firs extracting the catheter and/or the guide wire from the device.

In some embodiments, the connector is configured to allow the device to be detached from the bronchoscope without first extracting the catheter and/or the guide wire from the device

In some embodiments, the connector includes a luer lock plug configured to be positioned therein so as to allow for connection of a slip tip or a luer lock syringe.

In some embodiments, the catheter includes a luer lock entrance configured to be positioned therein so as to allow for connection of a slip tip or a luer lock syringe.

In some embodiments, the catheter can be used without the guide wire.

In some embodiments, the handle has a component configured to provide for data storage and for contactless communication. In some embodiments, the device stores a unique identifier that can be read in a contactless manner (e.g., through radio-frequency identification or near-field communication technology). In some embodiments, the handle includes an electronic device having general computing, data storage and wireless communication abilities. In some embodiments, a unique identifier is stored in the handle. In some embodiments, the handle unique identifier includes unique barcode that can be read by a barcode reader. In some embodiments, the barcode is stamped on the handle. In some embodiments, the barcode is stamped on the handle package. In some embodiments, the barcode is included in a product label.

In some embodiments, a radio opaque material includes, but is not limited to, materials including barium, iodine, or any combination thereof In some embodiments, two or more radio opaque materials are used in conjunction with one another.

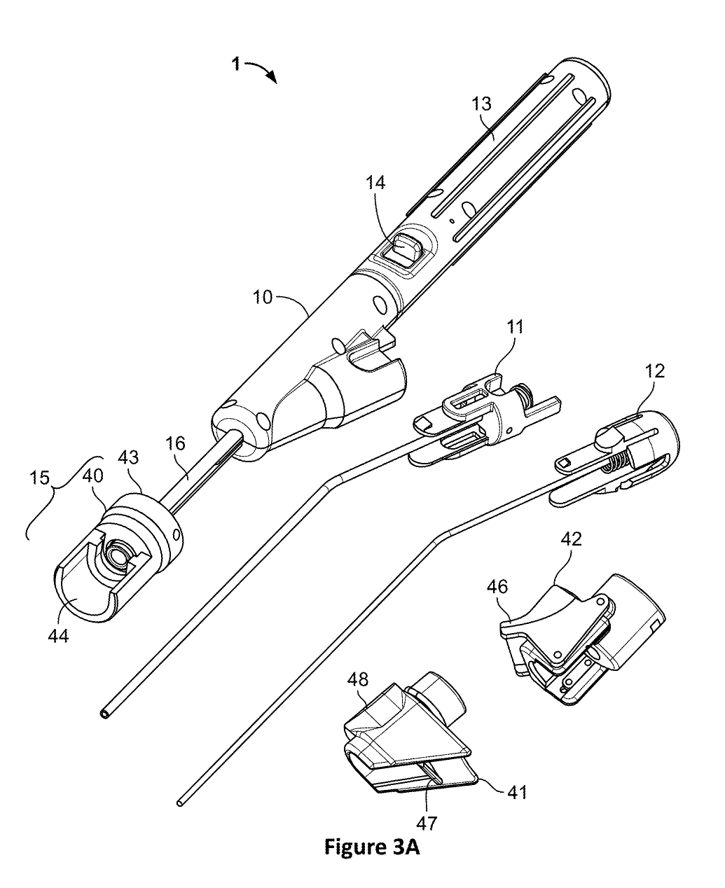

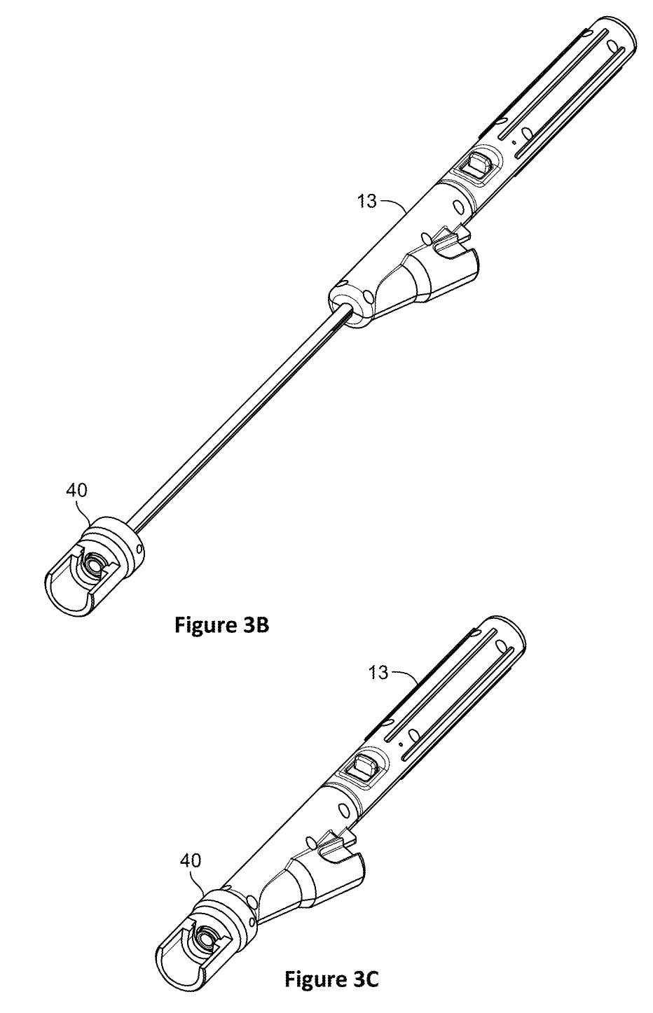

FIG. 3A shows the elements of an exemplary device 1. In some embodiments, the device 1 includes an applicator 10, a catheter 11 and a guide wire 12. In some embodiments, the applicator 10 includes a grip handle 13 that allows the user to pull, push, or rotate the grip handle 13 from a closed (retracted) position to an open (extended) position. In some embodiments, the applicator 10 includes an applicator shaft 16 that allows the grip handle 13 to slide along the applicator shaft 16 (i.e., along a longitudinal axis) while avoiding relative rotation between the applicator shaft 16 and the grip handle 13. In some embodiments, the applicator shaft 16 includes an internal passage that is configured to receive the catheter 11. Consequently, in some embodiments, rotation of grip the handle 13 causes the applicator shaft 16 to rotate therewith. In some embodiments, rotation of the grip handle 13 with respect to the shaft 16 can be selectively locked or unlocked, such that, when unlocked, the grip handle 13 is free to rotate with respect to the shaft 16. In some embodiments, the applicator 10 includes a connector element 15 that enables connection of the applicator 10 to any commercially used bronchoscope. In some embodiments, the connector element 15 includes a connector portion 40 that is permanently connected to the shaft 16. In some embodiments, the connector portion 40 is configured to connect the device 1 to a commercially used bronchoscope. In some embodiments, the connector portion 40 is connected to a bronchoscope by manually rotating swivel ring 43 in one direction, so as to move the swivel ring 43 toward and press a connector coupling 44 against the bronchoscope. In some embodiments, to detach the device 1 from the bronchoscope, the swivel ring 43 is manually rotated in the other direction, thereby moving the swivel ring 43 away from the connector coupling 44 and releasing pressure by the connector coupling 44 on the bronchoscope.

In some embodiments, the connector element 15 includes a connector portion 41 that can be detached from the shaft 16, and a connector portion 42 that can be detached from the shaft 16. In some embodiments, the connector portion 41 and the connector portion 42 may be connected to the shaft 16 by a snap 45 that is located at the distal end 32 of the shaft 16. In some embodiments, the connector portion 41 can be connected to a commercially available bronchoscope by sliding the connector portion 41 over an entrance port of the bronchoscope. In some embodiments, the connector portion 41 includes a connector slider 47 that is configured to slide over the entrance port of the bronchoscope and thereby lock the connector portion 41 to the bronchoscope. In some embodiments, the connector portion 41 includes a release button 48 that is operable to release the connector portion 41 from the bronchoscope. In some embodiments, the connector portion 42 includes a connector clasp 46. In some embodiments, the connector portion 42 can be connected to a commercially available bronchoscope by closing the connector clasp 46 against an entrance port of the bronchoscope. In some embodiments, the connector portion 42 can be removed from a commercially available bronchoscope by opening the connector clasp 46. In some embodiments, the connector portion 41 and the connector portion 42 can be connected to a bronchoscope in the absence of the applicator 10.

In some embodiments, the grip handle 13 includes a trigger 14 that is configured to lock the grip handle 13 at any position along its travel between its open and closed positions (e.g., along the applicator shaft 16). In some embodiments, the distal end of the shaft 16 is configured to act as a swivel, allowing the shaft 16 and the grip handle 13 to rotate with respect to the connector element 15 along the longitudinal axis to any desired angle.

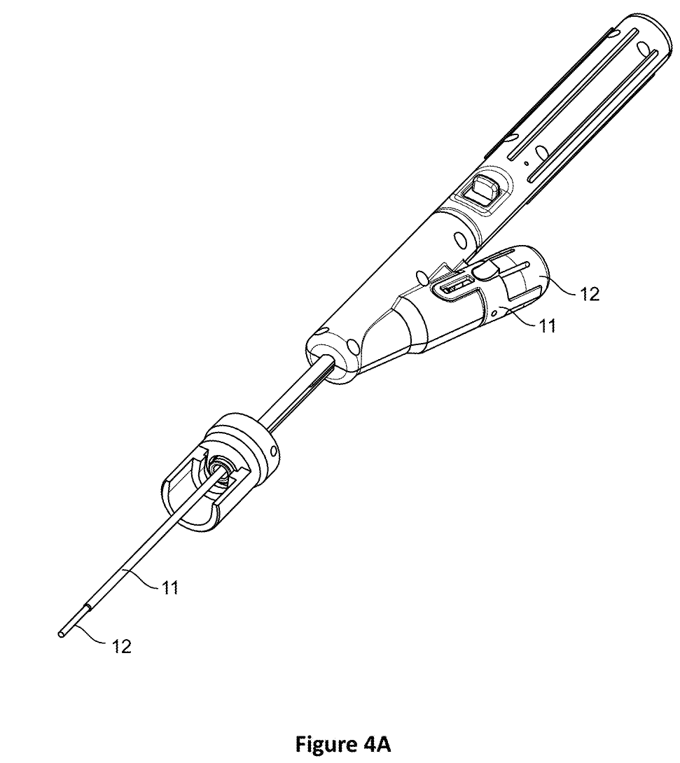

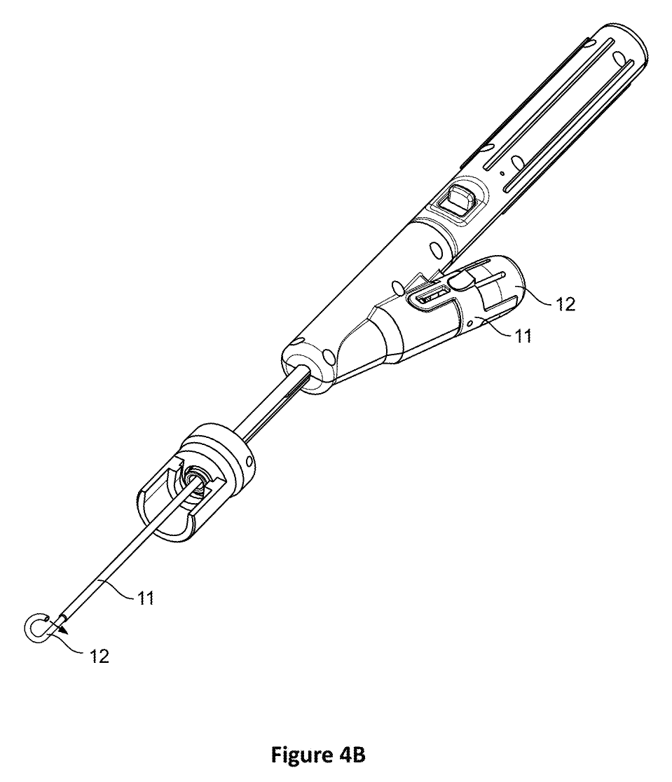

FIG. 3B shows the device 1 of FIG. 3A in its open (extended) position. The connector element 15 is extended distally from the grip handle 13. FIG. 3C shows the device 1 from FIG. 3B in its closed (retracted) position. The connector element 15 is in its closest proximity to the grip handle 13. FIG. 4A shows the device 1 of FIG. 3A, as configured with both the catheter 11 and the guide wire 12 connected to grip handle 13. FIG. 4B shows the device 1 of FIG. 4A, but with the guide wire 12 extended. In some embodiments, the device 1 includes a wire extraction button 33, which is configured to allow the guide wire 12 to be extended. In some embodiments, as shown in FIG. 4B, the guide wire 12 is flexible and can be positioned as needed.

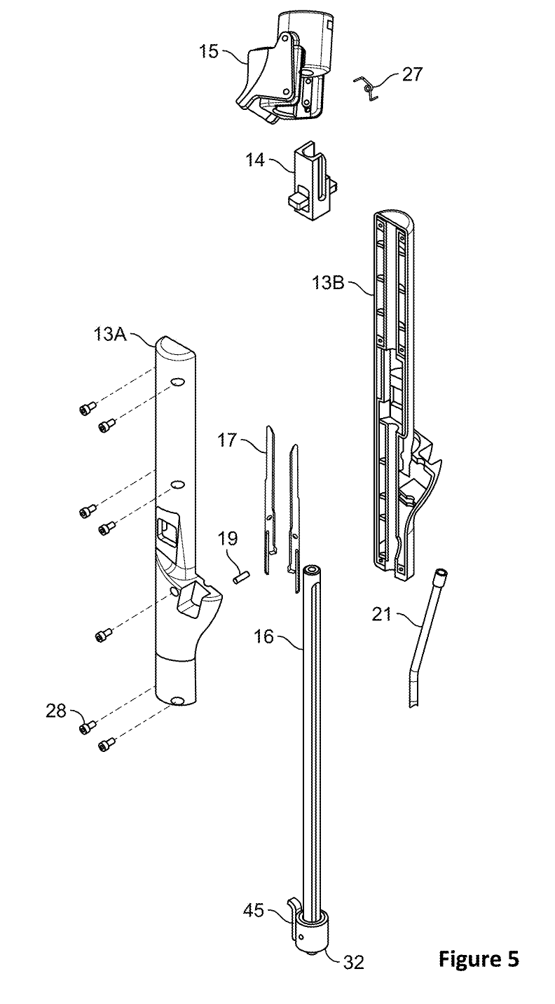

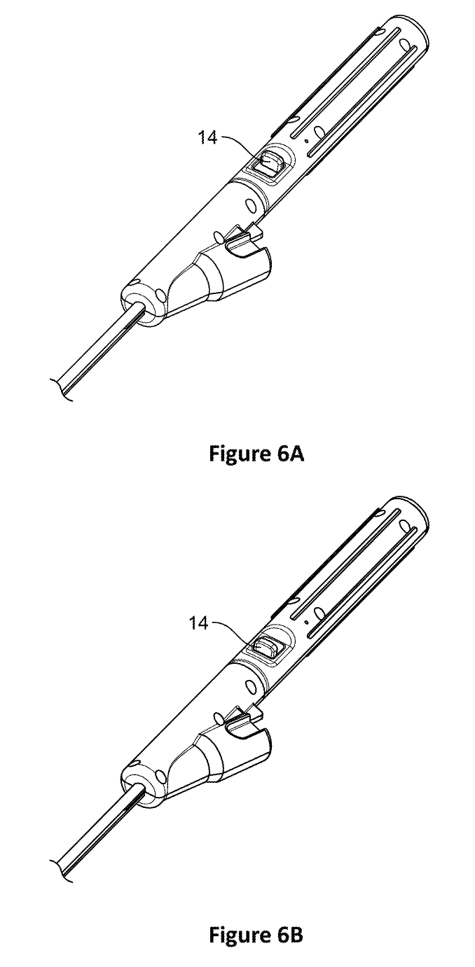

FIG. 5 shows an exploded view of the applicator 10. The grip handle 13 is divided into two side portions 13A and 13B. Screws 28 are configured to connect the two side portions 13A and 13B to one another. The applicator 10 includes a trigger 14, a lever 17, a hinge 19, and a spring 27, which will be described in detail with reference to FIGS. 6A and 6B below. The applicator 10 also includes an inlet tube 21 that is configured to receive the catheter 11.

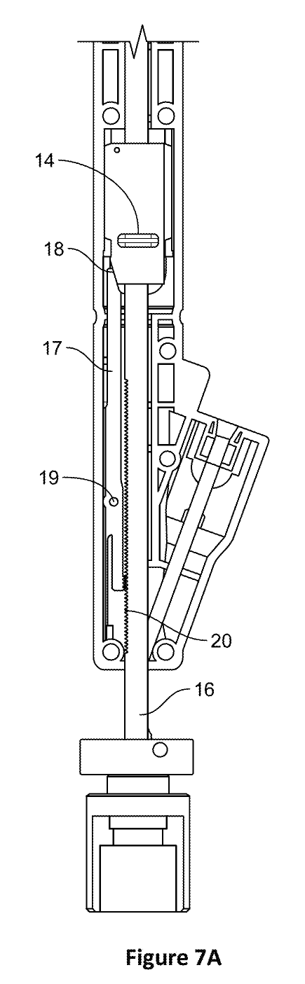

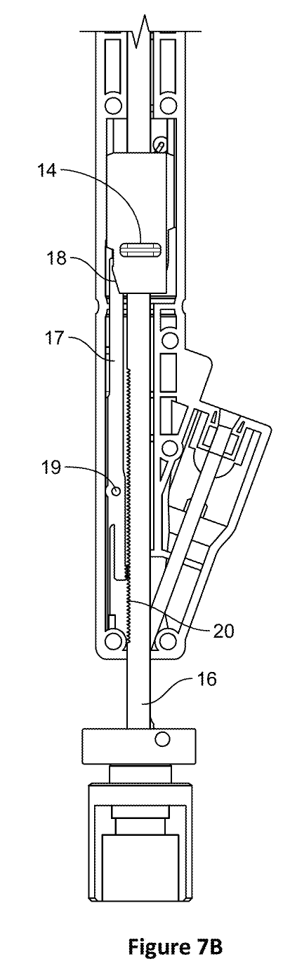

FIG. 6A shows the device 1 with the trigger 14 in its unlocked position, in which the shaft 16 is allowed to move with respect to the grip handle 13. FIG. 6B shows the device 10 with the trigger 14 in its locked position, in which the shaft 16 is allowed to move with respect to the grip handle 10. FIG. 7A shows a sectional view of the device 1 with the trigger 14 in its unlocked position. FIG. 7B shows a sectional view of the device 10 with the trigger 14 in its locked position. The device 1 includes a lock lever 17 that is pivotably engaged with a hinge 19. The shaft 16 has a grooved portion 20. The trigger 14 has an angled surface 18 that is configured to engage the lock lever 17 when the trigger 14 is in its locked position, and to disengage the lock lever 17 when the trigger 14 is in its unlocked position. When the angled surface 18 of the trigger 14 engages the lock lever 17 (e.g., as shown in FIG. 7B), the lock lever 17 pivots about the hinge 19 to a position such that the lock lever 17 engages the grooved portion 20 of the shaft 16, thereby preventing the shaft 16 from axial motion with respect to the grip handle 13. Conversely, when the angled surface 18 of the trigger 14 disengages the lock lever 17 (e.g., as shown in FIG. 7A), the lock lever pivots about the hinge 19 to a position such that the lock lever 17 does not engage the grooved portion 20 of the shaft 16, thereby allowing the shaft 16 to move axially with respect to the grip handle 13.

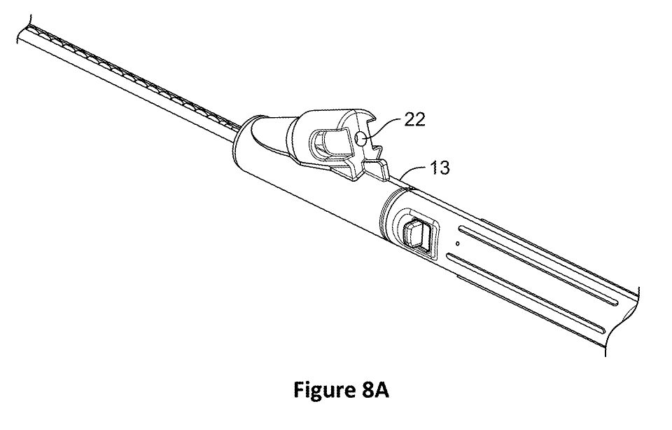

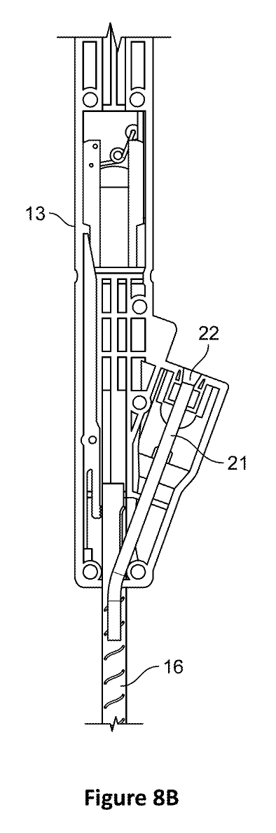

FIG. 8A shows a perspective view of the grip handle 13 in a direction facing toward the distal end of the grip handle 13. The grip handle 13 includes an inlet port 22 that allows insertion of the catheter 11 into the applicator 10. FIG. 8B shows a sectional view of a portion of the grip handle 13. The grip handle 13 includes an inlet tube 21 extending from inlet port 22 to the internal passage of the shaft 16, and configured to allow passage of the catheter 11.

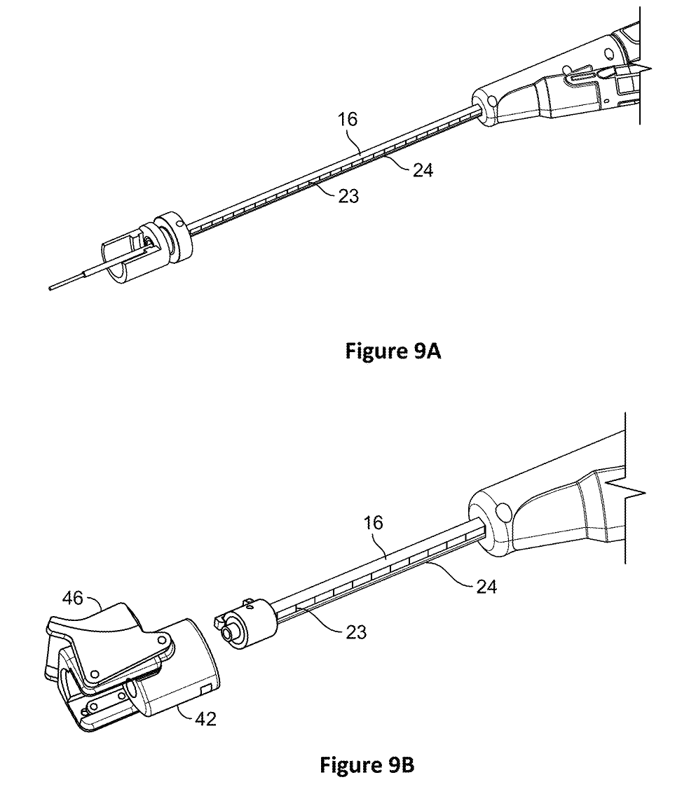

FIG. 9A and FIG. 9B show an opening 24 along the shaft 16 that allows the inlet tube 21 to slide from its extended position (i.e., as shown in FIG. 3B) to its closed position (i.e., as shown in FIG. 3C). In some embodiments, in order to prevent the catheter 11 and the guide wire 12 from buckling and protruding from the shaft 16 due to friction in a bronchoscope that is connected to the device 1, a polytetrafluoroethylene (“PTFE”, such as the material sold under the trade name TEFLON by DuPont) tube 23 is positioned inside the shaft 16 to act as a flexible barrier. In some embodiments, the PTFE tube 23 is positioned around the shaft 16 rather than inside the shaft 16. In some embodiments, rather than a PTFE tube 23, a spring, telescoping material or other flexible material that can withstand the buckling force is used. FIG. 9A shows the PTFE tube 23 in an extended position. FIG. 9B shows the PTFE tube 23 in a compressed position. In some embodiments, the PTFE tube 23 is connected to the connector element 15 at the distal end of the PTFE tube 23 and to the inlet tube 21 at the proximal end of the PTFE tube 23. As shown in FIG. 9B, when the connector element 15 is positioned proximate to the grip handle 13, the PTFE tube 23 is compressed.

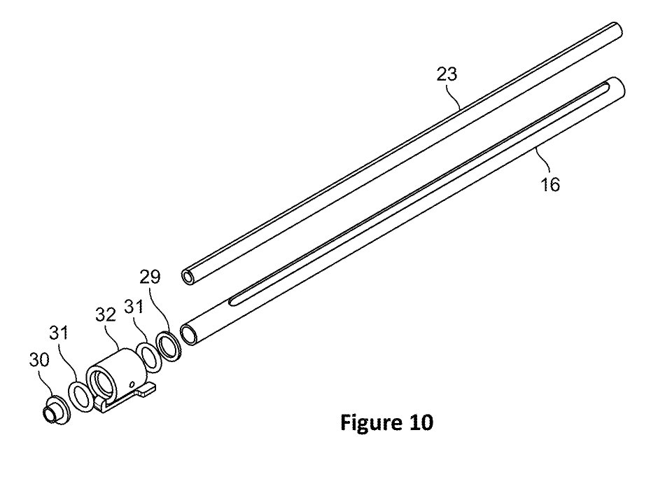

FIG. 10 shows an exploded view of the shaft 16. In some embodiments, the shaft 16 includes a swivel mechanism. In some embodiments, a PTFE tube 23 is positioned within the shaft 16 to act as a flexible barrier. In some embodiments, a shaft distal end 32 is free to rotate with respect to the shaft 16. In some embodiments, the swivel mechanism also includes two washers 29 and 30 and two o-rings 31 that provide control to the rotation. In some embodiments, the shaft distal end 32 is configured to be attached to the connector 15.

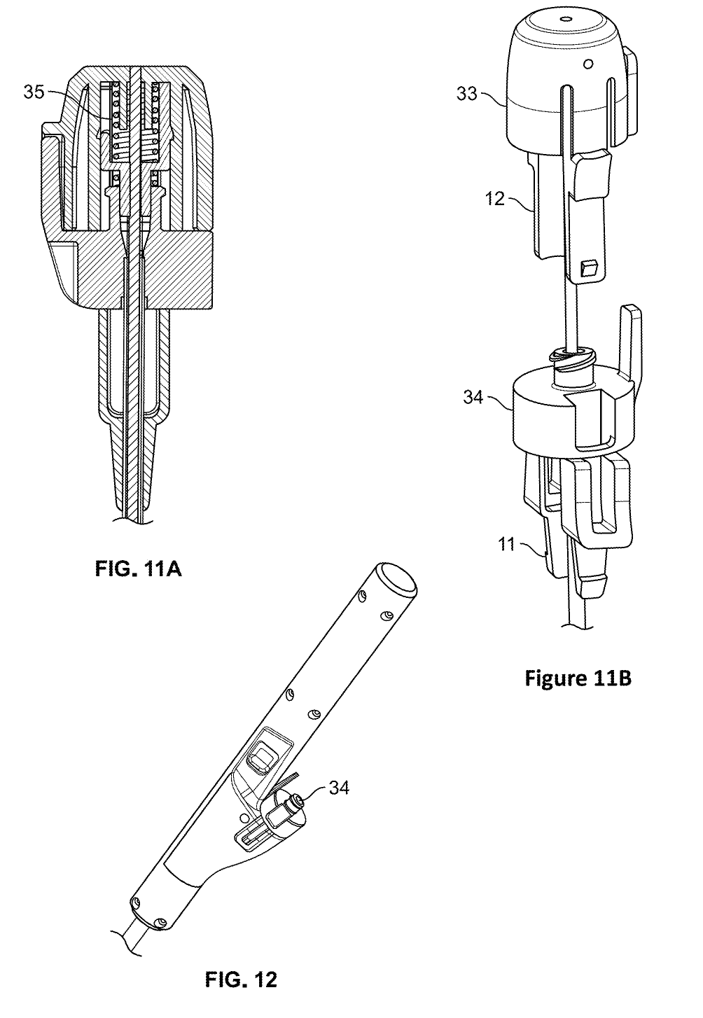

FIG. 11A and FIG. 11B show a sectional view and an exploded view, respectively, of a wire extraction button 33. In some embodiments, the wire extraction button 33 presses against a spring 35, which biases the wire extraction button 33 to a position in which the wire extraction button 33 restrains movement of the guide wire 12. In some embodiments, the wire extraction button 33 is removably coupled to a sheath luer lock entrance 34, which is configured to allow connection to a syringe. In some embodiments, the wire extraction button 33 can be removed to expose the sheath luer lock entrance 34. FIG. 12 shows the proximal portion of the applicator 10 with the sheath luer lock entrance 34 exposed.

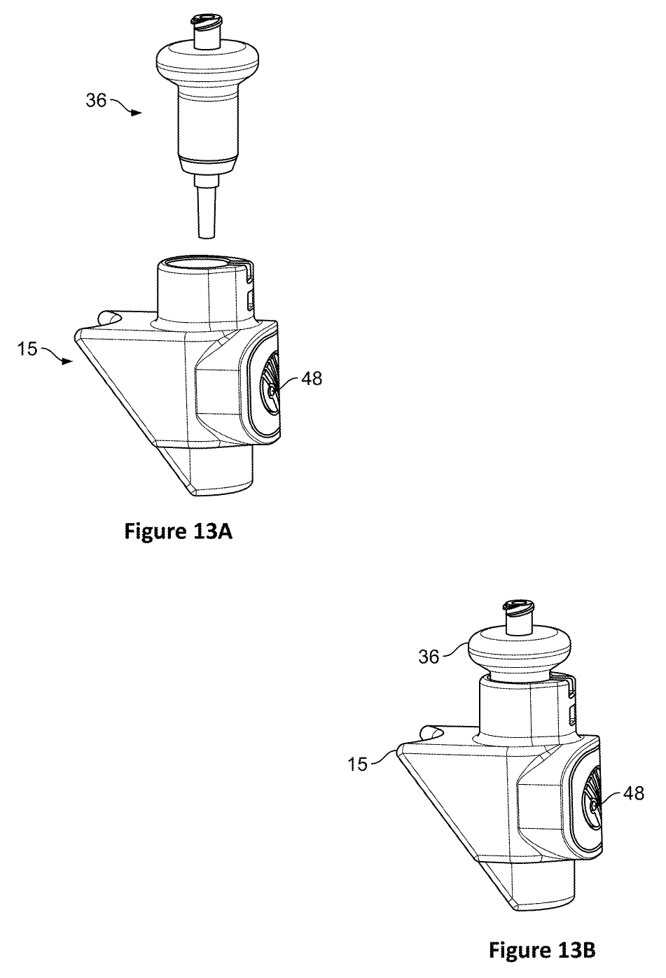

FIG. 13A shows a luer lock plug 36, which can be connected to the connector portion 41 or the connector portion 42 to allow a syringe connection to the connector 15. FIG. 13B shows the luer lock plug 36 as connected to the connector 15.

In some embodiments, the present invention relates to a radio opaque pattern on a device, where the radio opaque pattern can be visualized by a user (e.g., a doctor, etc.) and used to identify the specific portion of the device visible on the x-ray image, e.g., by correlating portions of the device with the observed density of the radio opaque material. In some embodiments, the radio opaque material is positioned on the catheter 11 of the device 1. In some embodiments, the radio opaque material is positioned on the guide wire 12 of the device 1. In some embodiments, the radio opaque material is positioned on both the catheter 11 and the guide wire 12 of the device 1, which cooperate to produce a combined “effective” pattern of radio opaque material on the device 1.

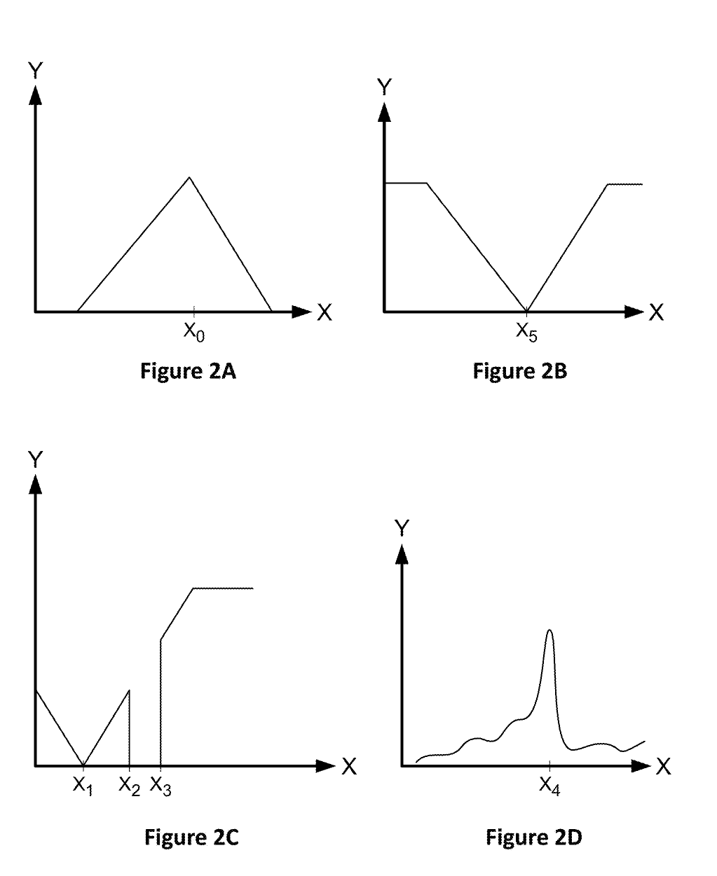

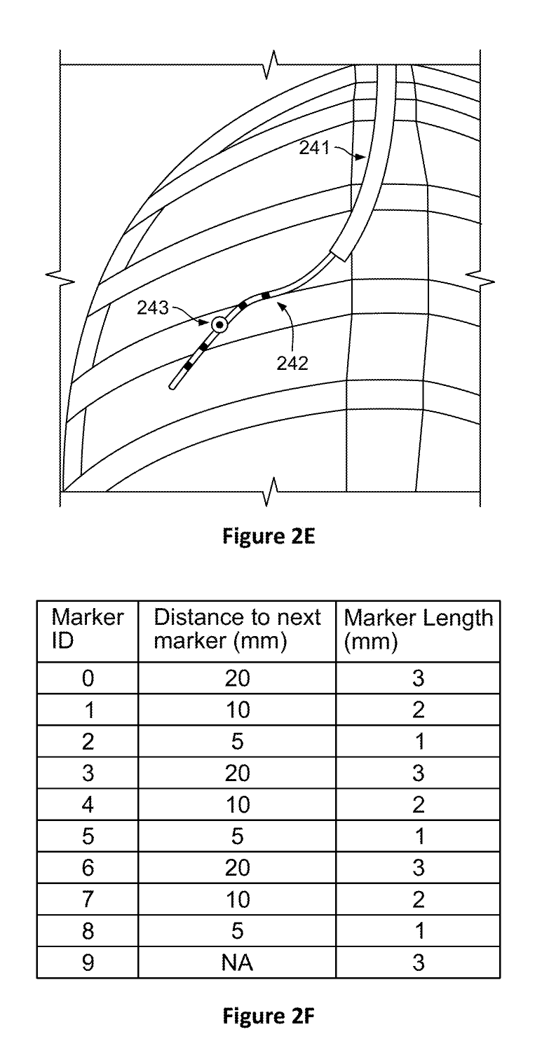

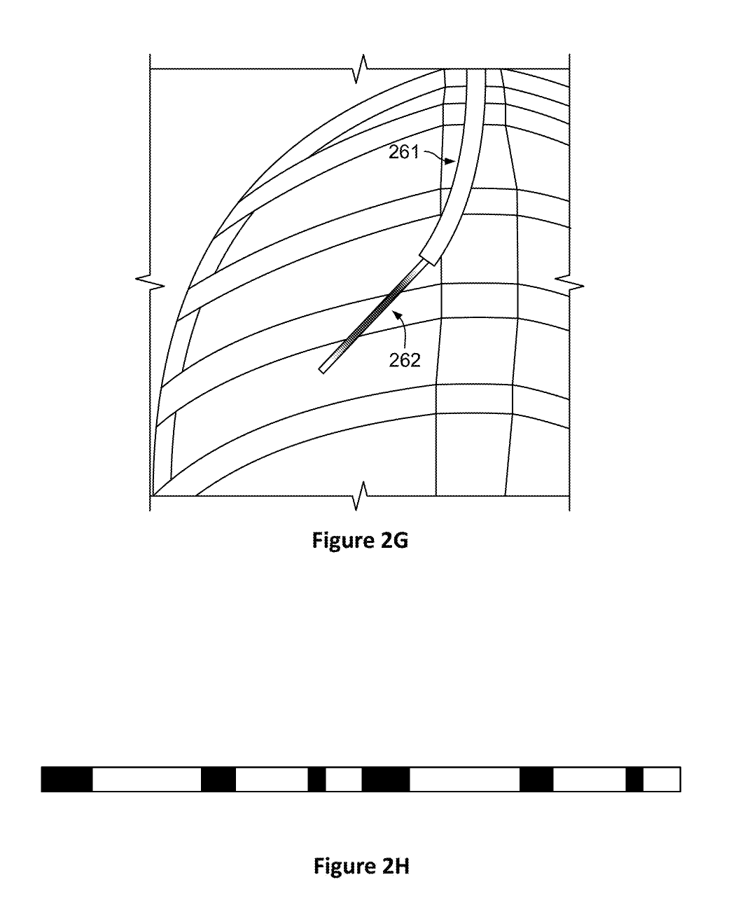

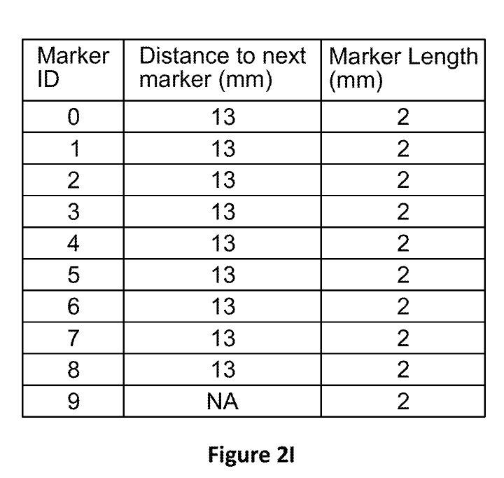

In some embodiments, the device 1 of the current invention has a radio opaque material positioned in a pattern which can be observed (e.g., but not limited to, using X-ray images of the device), where the pattern has been manufactured by applying variable amount(s) of radio opaque material along the device. In some embodiments, the correlation between the function of radio opaque material density along the device and the function of grayscale intensity in the x-ray image allows the detection of a specific portion of the device on the fluoroscopic image in spite of partial occlusion by other radio opaque objects on the image. In some embodiments, the higher density of radio opaque material in the device results in lower gray-scale intensities visualized by the X-ray image and vice versa. FIG. 2A shows a plot of radio opaque material density along the length of an embodiment of device (Y axis), as plotted against the length of the device (X axis). FIG. 2B shows one-dimensional gray scale levels (Y axis) of a device with material density as shown in FIG. 2A, as imaged by a fluoroscope along the length of the device (X axis). Taken together, FIGS. 2A and 2B show that the density of the radio-opaque material is correlated with gray-scale image function.

FIG. 2C shows one-dimensional gray scale levels (Y axis) of a partial device protruding from a bronchoscope (as compared to FIG. 2B, which illustrates the full image of the device), imaged by a fluoroscope along to the length of the device (X axis). The zero value between positions x2 and x3 along the X axis illustrates an occlusion that blocks the X-ray radiation in this interval. FIG. 2D shows the absolute value of the correlation function between the partially imaged device (i.e., as shown in FIG. 2C) and the density of the radio opaque material (i.e., as shown in FIG. 2A). The position of the peak in FIG. 2D can be utilized to calculate the translation between pixels in FIG. 2C and 3 dimensional model coordinates in FIG. 2A. FIG. 2E shows a representation of an X-Ray image showing a bronchoscope 241 and device 242 (e.g., the device 1) with radio opaque material, as positioned within the chest of a patient. At position 243, the device 242 is occluded by an ECG patch.

In some embodiments, the radio opaque material is arranged along the device 1 in a pattern. In some embodiments, the pattern includes differently sized rings extending around the device. In some embodiments, the pattern includes rings irregularly spaced along the device. FIG. 2F shows a table showing a first pattern comprised of rings of radio opaque material located at different spacing from one another and having different lengths. FIG. 2I shows a table showing a second pattern comprised of rings of radio opaque material located at different spacing from one another and having different lengths. It will be apparent to those of skill in the art that the specific patterns represented by FIG. 2F and FIG. 2I are only exemplary and that other patterns are possible.

FIG. 2G shows a representation of an X-Ray image showing a bronchoscope 261 and device 262 (e.g., the device 1) having radio opaque material that is patterned as shown in FIG. 2A. FIG. 2H shows an illustration of a pattern of radio opaque material containing rings of variable size, placed in positions at varying intervals along the outer portion of a device (e.g., the device 1).

In a non-limiting example, when a portion of a pattern of radio opaque material is visible, a user can calculate the one-dimensional translation (e.g., correlation) between the imaged pattern and the density function. The relation between the radio opacity of the device and the gray-scale levels can be used for this purpose. In another non-limiting example, a user can use a template matching method that searches for the highest correlation between the gray-scale scale levels of the visible segment of the device in the image and the radio opaque density profile of the device. Such a method is robust to occlusion and noise caused by objects that are behind or above the device with respect to the projection direction from an X-ray tube to an image intensifier. In some embodiments, FIG. 2D shows an exemplary correlation function between the device's partial image as shown in FIG. 2C and the device's pattern of radio opaque material density as shown in FIG. 2A. For instance, the translation between the density function at point x0 in FIG. 2A to the pixel gray-scale level at point x1 on FIG. 2C corresponds to the peak position at the point x4 in the correlation function shown in FIG. 2D. As a result, although the device as represented by FIG. 2C is partially visible and partially occluded in the area between points x2 and x3, it is possible to perform device localization on the image and correlate each pixel of the visible device, as represented by FIG. 2D to the known model for the device, as represented by FIG. 2A.

In some embodiments, a unique radio opaque pattern is manufactured through attaching radio opaque rings of variable size to the device at specific positions along the device's longitude direction axis, as illustrated by FIG. 2H. The unique radio opaque pattern assists a user in estimating the transformation function between the imaged device's pixels and predesigned device model for manufacturing. This transformation function can be estimated by finding a function that satisfies the constraints imposed by the different marker sizes and locations on the device. A non-limiting example for such design, which is robust to occlusion of several markers on x-ray image, is provided in FIG. 2F.

In some embodiments, a medical image (e.g., an X-ray image) of at least a portion of a body of patient with the device 1 (i.e., which includes the radio opaque material) positioned within the body of the patient can be analyzed to determine the depth of the device 1 within the body based on knowledge of the positioning of the radio opaque material. In some embodiments, the current invention relates to a method to recover 3-dimensional depth information in such cases, where due to occlusions and noise of the 2-dimensional image as an input, such as X-ray image or video image sequence, some markers may not be detected, by means of unique pattern on the device as shown, for example, in FIG. 2A. The occlusion and noise of the input image or video image sequence may be caused by occlusion of medical devices, high density tissue such as ribs, patient pace makers, ECG cables, etc. as illustrated by FIG. 2E.

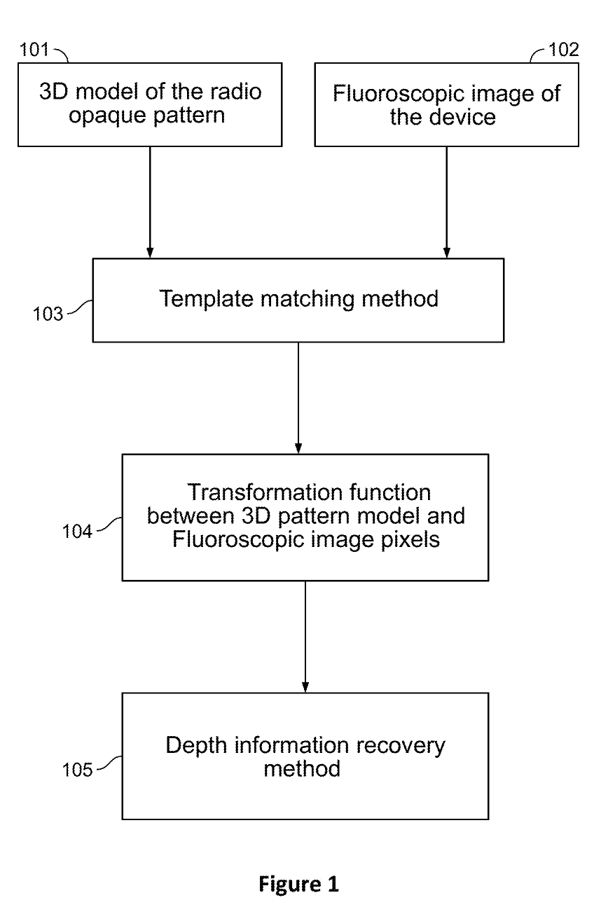

FIG. 1 shows a flowchart of a process for determining the depth of an exemplary device (e.g., the device 1 of FIG. 3A). The process receives, as inputs, a density model (101) of the radio opaque material along the device (e.g., the information shown in FIG. 2A) and fluoroscopic image data (102) showing the device positioned within the patient's body. A transformation function (104) between the model and the image pixels is calculated using a template matching method (103). In some embodiments, the template matching method is performed as described above with reference to FIGS. 2A-2D. The transformation function is used for depth information recovery (105).

In some embodiments, the depth of the device can be calculated from a single image based on prior knowledge the physical dimensions of the specific radio opaque pattern. For instance, given the known physical distance between two points that are identified and located in the intra operative image, one can determine the relative depth between these two points. In some embodiments, such a technique for determining relative depth is carried out as described in International Patent Application Publication No. WO/2015/101948, the contents of which are incorporated herein by reference in their entirety. More particularly, in some embodiments, a device (e.g., the device 1) or a portion thereof (e.g., the portion between two of the stripes shown in FIG. 2H) having a known length “L3” and located in three-dimensional space within a patient's body is projected into an imaging plane to create a projection image including such a device. The observed (i.e., projected) length of the same device (or device portion) in the two-dimensional imaging plane is “L2”. As shown in FIG. 12 of International Patent Application Publication No. WO/2015/101948, an angle α of the device (or device portion) in space can be determined by solving the equation L2=L3 cos α. The relative depth D between the two ends can then be determined by calculating D=L3 sin α.

In some embodiments, the depth of the device can be calculated using the methods described in International Patent Application Publication No. WO/2017/153839, the contents of which are incorporated herein by reference in their entirety. In some embodiments, such determination is performed according to the following process. In some embodiments, the device is imaged by an intraoperative device and projected to an imaging plane. In some embodiments, a predefined distance “m” between two radiopaque regions “F” and “G” on the device (e.g., two of the stripes shown in FIG. 2H) is considered as an input. In some embodiments, point “F” results from a projection of two possible 3D locations A and B, having different depth from one another. In some embodiments, point “G” results from a projection of two possible depth locations C and D, having different depth from one another, and where C corresponds to A and D corresponds to B. In some embodiments, 3D distances between the back-projected location pairs AC and BD are measured. In some embodiments, the 3D distances AC and BD are compared to the distance “m”, and either points A and C or points B and D are selected based on the best fit. In some embodiments, the depth is that corresponding to the selected pair of locations.

In some embodiments, the depth recovery can be performed using a combination of a known patient anatomy and pose estimation approach. In some embodiments, the knowledge of the unique radio opaque pattern can be combined with the knowledge of the patient's anatomical bronchial tree (e.g., as extracted from the pre-operative image) and the knowledge of the current pose of the imaging device relative to the patient (e.g., a point of view that allows projecting 3D information from a pre-operative image to the current image acquired from the imaging device). Since an instrument is located inside a discrete anatomical space, the current pose estimation information can be used to limit the possible solutions. Furthermore, the matching between the instrument location and possible anatomical location on the bronchial tree can be recovered by solving an optimization problem with respect to the following parameters: an assumption of the anatomical location of the tool, a pose estimation, and potential 3d anatomy changes. In some embodiments, such an approach is described in greater detail in International Patent Application Publication No. WO2015/101948.

In some embodiments, the depth estimation can be performed from a sequence of two or more images by (a) finding corresponding points between views, for example, by tracking or matching by visual similarity; (b) finding pose relative differences using, for example, a jig, human anatomy, or any other pose estimation algorithm (e.g., those described in International Patent Application Publication No. WO/2017/153839); and (c) reconstructing three-dimensional information of the matching points from multiple images with known poses using methods that are known in the art (e.g., triangulation, a stereo corresponding point based technique, a non-stereo corresponding contour method, a surface rendering technique, etc.).

In some embodiments, the device provides increased maneuverability inside a body cavity, e.g., but not limited to, bronchial airways, compared to typical methods. In some embodiments, the device is as seen in the non-limiting example shown in FIGS. 3A-13B. In some embodiments, the exemplary device allows increased accuracy while navigating with one hand and supports the standard diagnostic and therapeutic device's entrance from the other. In some embodiments, the guide wire is pre-curved. In some embodiments, the catheter is pre-curved. In some embodiments, both the guide wire and the catheter are pre-curved. In some embodiments, the guide wire is straight. In some embodiments, the catheter is straight. In some embodiments, both the guide wire and the catheter are straight. In some embodiments, the guide wire is configured to be bent as needed. In some embodiments, the catheter is configured to be bent as needed. In some embodiments, both the guide wire and the catheter are configured to be bent as needed. In some embodiments, the guide wire is configured to protrude past the tip of the catheter, while adding extra bending to the device. This feature allows for increased maneuverability of the device during the navigation inside the lung.

In some embodiments, the device including the radio opaque material includes an endoscope, an endo-bronchial tool, and/or a robotic arm.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

What is claimed is:

1. A device configured to be attached to a bronchoscope, the device comprising:
an applicator having a proximal end, a distal end, and an internal channel extending from the proximal end to the distal end;
a shaft having a proximal end, a distal end, and an internal channel extending from the proximal end to the distal end, the shaft being configured to be slidably received within the internal channel of the applicator;
a catheter configured to be positioned within the internal channel of the shaft;
a guide wire positioned within the catheter;
a connector configured to be attached to the distal end of the applicator, configured to engage a bronchoscope, and configured so as to be rotatable with respect to the shaft;
a handle attached to the proximal end of the applicator, the handle comprising a trigger operable to selectively lock or unlock sliding motion of the shaft with respect to the applicator; and
a radio opaque material attached to an outer portion of the device, the radio opaque material being positioned in a predetermined pattern.

2. The device of claim 1, wherein the pattern is non-uniform.

3. The device of claim 1, wherein the pattern includes the radio opaque material having a first density at a first location and a second density at a second location, the first and second densities being different from one another.

4. The device of claim 1, wherein the radio opaque material is positioned (a) on the catheter, (b) on the guide wire, or (c) on both the catheter and the guide wire.

5. The device of claim 1, wherein the proximal end of the applicator includes a luer lock entrance.

6. The device of claim 5, wherein the connector includes a luer lock plug that is connected to the luer lock entrance of the proximal end of the applicator.

7. The device of claim 1, wherein the guide wire is either flexible, rigid, pre-curved, and or configured to be curved.

8. The device of claim 1, wherein the catheter includes a pull wire that is configured to control a curvature of the guide wire.

9. The device of claim 1, wherein the grip handle is configured to rotate with respect to the shaft.

10. The device of claim 1, further comprising a polytetrafluoroethylene tube positioned within the shaft and configured to guide movement of the catheter.

11. A method for medical imaging, comprising:
providing a bronchoscope;
providing a device configured to be attached to the bronchoscope, the device including
an applicator having a proximal end, a distal end, and an internal channel extending from the proximal end to the distal end;
a shaft having a proximal end, a distal end, and an internal channel extending from the proximal end to the distal end, the shaft being configured to be slidably received within the internal channel of the applicator;
a catheter configured to be positioned within the internal channel of the shaft;
a guide wire positioned within the catheter;
a connector configured to be attached to the distal end of the applicator, configured to engage a bronchoscope, and configured so as to be rotatable with respect to the shaft;
a handle attached to the proximal end of the applicator, the handle comprising a trigger operable to selectively lock or unlock sliding motion of the shaft with respect to the applicator; and
a radio opaque material attached to an outer portion of the device, the radio opaque material being positioned in a predetermined pattern;
attaching the device to the bronchoscope;
placing the bronchoscope within a body cavity of a body of a patient;
obtaining at least one medical image of at least a portion of the body of the patient, the at least a portion including the body cavity; and
determining a depth of the device within the body based on at least the predetermined pattern and the at least one medical image.

12. The method of claim 11, wherein the medical image is an X-ray.
